(19) United States
(12) Patent Application Publication
(10) Pub. No.: US 2007/0038229 A1
(43) Pub. Date: Feb. 15, 2007

(54) Flexible endoscopic knot tying apparatus

(75) Inventors: Roger A. de la Torre, Wentzville, MO (US); Matthew P. LaConte, Maryland Heights, MO (US)

Correspondence Address: THOMPSON COBURN, LLP, ONE US BANK PLAZA, SUITE 3500, ST LOUIS, MO, 63101, US

(73) Assignee: TowerTech Research Group

(21) Appl. No.: 11/203,267

(22) Filed: Aug. 12, 2005

(51) Int. Cl. A61B 17/10

(52) U.S. Cl. 606/139

(57) ABSTRACT

A flexible endoscope has an elongate, narrow tube with at least three distinct interior bores. A first of the bores is provided to accommodate surgical devices typically provided in endoscopes. The second and third bores of the flexible tube mount a flexible suture holder and a flexible grasper in the interior of the tube. The suture holder and the grasper are movable through their respective bores relative to the endoscope tube. A distal end portion of the suture holder is curved, whereby extending the suture holder from the endoscope tube and extending the grasper from the endoscope tube will cause a distal end of the suture holder to curve to a position where it intersects with the distal end of the grasper. A length of suture is removably mounted to the distal end of the suture holder. With the suture holder and the grasper moved to their extended positions relative to the endoscope tube, the surgical grasper is in position to grasp and remove the length of suture from the distal end of the suture holder. A pre-tied knot on the length of suture enables the apparatus to be used in endoscopic procedures involving legation of body tissue.

### BACKGROUND OF THE INVENTION

(1) Field of the Invention

The present invention pertains to a flexible endoscope having an elongate, narrow tube with at least three distinct interior bores. A first of the interior bores is provided to accommodate surgical devices typically provided in endoscopes. The second and third interior bores mount a flexible suture holder and a flexible grasper in the interior of the tube. The suture holder and the grasper are movable through their respective bores relative to the endoscope tube. A distal end portion of the suture holder is curved, whereby extending the suture holder from the endoscope tube and extending the grasper from the endoscope tube will cause a distal end of the suture holder to curve to a position where it intersects with the distal end of the grasper.

A length of suture is removably mounted to the distal end of the suture holder. With the suture holder and the grasper moved to their extended positions relative to the endoscope tube, the surgical grasper is in position to grasp and remove the length of suture from the distal end of the suture holder. A pre-tied knot on the length of suture enables the apparatus to be used in endoscopic procedures involving ligation of body tissue.

(2) Description of the Related Art

Flexible endoscopes have been used for many years in the examination and treatment of the body's internal tissues and organs. The basic construction of the prior art flexible endoscope includes an elongate flexible tube having a hollow interior bore extending through the entire length of the tube. The endoscope tube typically contains devices that allow a surgeon to view images of the body's interior by insertion of the tube through small incisions in the body. The tube is constructed with a very small exterior diameter, for example less than 5 mm, to enable use of the flexible endoscope in minimally invasive procedures where the tube is inserted through a very small body incision that heals quickly after the procedure.

The viewing devices of an endoscope often include one or more optic fibers that extend through the length of the tube interior bore. The optic fiber or fibers are secured stationary in the interior bore of the tube. One of the optic fibers is employed in transmitting illuminating light through the endoscope to the distal end of the flexible tube. The light transmitted from the tube distal end by this optic fiber illuminates the area where the surgical procedure is being performed. The other optic fiber is used to transmit an image of the illuminated surgical site back through the endoscope to a viewing lens at the proximal end of the endoscope, or a separate viewing monitor.

Endoscopes are viewing instruments only and are used with other, separate surgical instruments in performing endoscopic procedures. Additional incisions are made in the body tissue and trocars are inserted through the additional incisions to provide access for the additional surgical instruments to the surgical site in the body. In such minimally invasive surgical procedures, the objective is to reduce the size and number of incisions made in the body tissue to access the surgical site. Any reduction in the number of incisions in the body tissue needed in performing a surgical procedure reduces the surgical trauma and benefits the minimally invasive surgical procedure.

### SUMMARY OF THE INVENTION

The flexible endoscope of the present invention benefits minimally invasive surgical procedures by providing a flexible endoscope that is not only capable of performing the surgical view functions currently performed by endoscopes, but also provides flexible instruments that are capable of performing knot tying procedures, for example endoscopic ligation procedures. By providing ligating instruments in the flexible endoscope, the number of body incisions needed to perform an endoscopic surgical procedure can be reduced.

The endoscope of the present invention comprises an elongate flexible tube having opposite proximal and distal ends. The interior of the tube is constructed with three, separate interior bores that extend the length of the tube. A first of the interior bores is dedicated to securely mounting surgical viewing devices typically provided in endoscopes. For example, an illumination optic fiber and a viewing optic fiber may extend along the length of the flexible tube in the first interior bore. The second and third interior bores are separated from the first interior bore and are provided for mounting the novel instruments of the endoscope of the invention that are used in performing knot tying ligation steps.

An elongate suture holder is mounted in the second bore for reciprocating movement between first and second positions of the suture holder relative to the endoscope tube. A distal end of the suture holder is constructed to removably hold a length of suture. Movement of the suture holder through the tube second bore produces movement of the suture holder distal end and the length of suture between a first position, where the suture holder distal end and the length of suture are positioned adjacent the distal end of the endoscopic tube, and a second position where the suture holder distal end and the length of suture are extended outwardly from the tube distal end.

An elongate, flexible grasper is mounted in the third bore for reciprocating movement between first and second positions of the grasper relative to the endoscope tube. A distal end of the grasper has a pair of jaws that are selectively opened and closed to perform grasping operations. In the first position of the grasper, the grasper distal end is positioned adjacent the tube distal end. In the second position of the grasper, the grasper distal end is extended outwardly from the tube distal end.

The suture holder distal end has a curved portion that curves into the path of movement of the grasper when the suture holder is moved to its second position relative to the endoscope tube. With the suture holder in its second position, when the grasper is moved to its second position the grasper distal end is positioned to operatively grasp the length of suture held by the suture holder distal end. On movement of the grasper away from its second position after grasping the length of suture, the grasper removes the suture from the suture holder distal end.

The movements of the suture holder and grasper relative to the endoscope tube enable passing the length of suture around body tissue to be ligated. By providing a knot in the length of suture and around the surgical grasper, the suture holder and surgical grasper can be manipulated to pass the length of suture around body tissue to be ligated, and then tighten the suture around the ligated tissue by tightening the knot in the length of suture.

### BRIEF DESCRIPTION OF THE DRAWINGS

Further features of the invention are set forth in the following detailed description of the preferred embodiment of the invention, and in the drawing figures wherein:

FIG. 1 is a view of the flexible endoscope of the present invention;

FIG. 2 is a partial, enlarged view of opposite proximal and distal end portions of the endoscope of FIG. 1 showing the initial steps of a ligating method performed by the endoscope;

FIG. 3 is a view similar to FIG. 2, but showing further steps of the ligating method performed by the endoscope;

FIG. 4 is a view similar to FIG. 3, but showing a further step in the ligating method; and, FIG. 5 is a view similar to FIG. 4 showing the completion of the ligating method.

### DETAILED DESCRIPTION OF THE PREFERRED EMBODIMENT

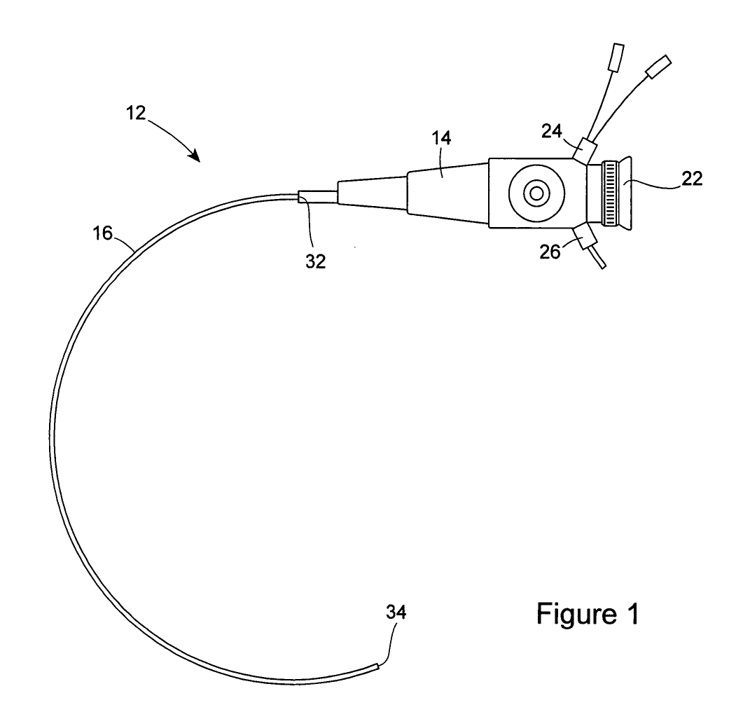

The flexible endoscope (12) of the present invention is shown in FIG. 1. It is contemplated that the endoscope (12) may have many of the features of prior art endoscopes, with the novel addition of the surgical devices to be described. Thus, the overall endoscope construction shown in FIG. 1 is only one example of the possible construction of the endoscope (12) of the present invention. Because endoscopes are known in the prior art, the known construction features of the endoscope (12) of the invention shown in FIG. 1 will be described only generally.

The endoscope (12) is basically comprised of a control housing (14) and an elongate tube (16). These features are found in many prior art endoscopes. The materials employed in constructing these features of the invention are the same materials commonly employed in the construction of prior art endoscopes.

The control housing (14) shown in FIG. 1 is provided with a viewing lens (22). The lens (22) is used by the surgeon in viewing surgical procedures using the endoscope. In variant embodiments of the invention, the endoscope may be connected electronically with a viewing monitor whereby the surgeon views the surgical procedure by observing movements of the endoscope on a viewing screen of the monitor. The control housing (14) is also shown with a plurality of ports (24, 26) extending from the housing. These ports may be used in adding additional features to the endoscope (12), for example aspiration and/or irrigation features.

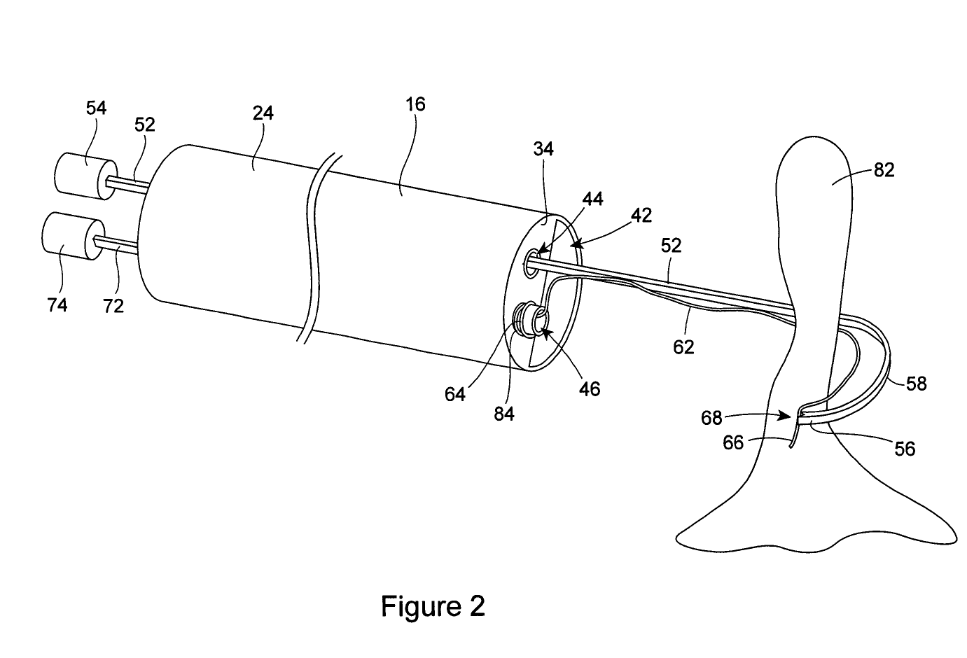

The elongate tube (16) has a flexible length with opposite proximal (32) and distal (34) ends. The tube (16) has a reduced exterior diameter dimension, for example less than 5 mm, that is typical in flexible endoscopes. The tube's exterior surface is smooth and the exterior diameter is constant along the length of the tube. As shown in FIGS. 2-5, the tube (16) is constructed with three, separate interior bores that each extend the length of the tube between the proximal end (32) and the distal end (34).

A first interior bore (42) of the flexible tube (16) is dedicated to securely mounting surgical viewing devices typically employed in endoscopes. This first interior bore (42) extends the entire length of the tube (16) and communicates with the interior of the control housing (14). Thus, the first tube bore (42) and the control housing (14) provide accommodation for surgical viewing devices typically employed in endoscopic surgery. To provide ample room for these devices, the first interior bore (42) occupies a majority of the cross sectional area of the interior of the tube (16).

A second interior bore (44) is provided in the interior of the tube (16) and extends the entire length of the tube between the opposite proximal (32) and distal (34) ends. As shown in FIGS. 2-5, the second interior bore (44) is separated from the first interior bore (42). The second interior bore (44) emerges from the tube proximal end (32) into the control housing (14) whereby the novel surgical instruments of the invention to be described can pass through the tube (16) into the control housing (14).

A third interior bore (46) also extends through the length of the elongate tube (16) between the tube proximal (32) and distal (34) ends. As shown in FIGS. 2-5, the third interior bore (46) is separated from the second interior bore (44) and the first interior bore (42). The third interior bore (46) is also spaced at a fixed, predetermined transverse position relative to the second interior bore (44), as will be explained.

The elongate flexible tube (16) is constructed so that it is freely flexible along its length. In addition, the interior bores (42, 44, 46) of the tube (16) are constructed in a manner where each bore is surrounded by an interior wall that flexes with the flexing movement of the elongate tube (16) without the cross-sectional areas of each of the bores collapsing. This enables unobstructed access through each of the interior bores (42,44,46) throughout the entire flexing movement of the tube (16).

A narrow elongate suture holder (52) is mounted in the tube's second bore (44) for reciprocating movements of the suture holder through the bore. The suture holder (52) has a narrow flexible length that extends between opposite proximal (54) and distal (56) ends of the suture holder. The length of the suture holder (52) extending through the second bore (44) flexes with the flexing movement of the tube (16). The length of the suture holder (52) is slightly longer than the length of the endoscope (12), whereby the suture holder proximal end (54) projects from a port (24) of the control housing (14) with the suture holder distal end (56) being positioned adjacent and inside the distal end of the flexible tube (16). The suture holder proximal end (54) is shown projecting from the control housing (14) where it can be manually manipulated by the surgeon to move the suture holder (52) between first and second positions relative to the tube (16). As an alternative to the manual manipulation of the suture holder proximal end (54), the suture holder proximal end could be connected to a mechanism on the control housing (14) that is manipulated by the surgeon to cause the suture holder (52) to move between its first and second positions relative to the tube (16). In the first position of the suture holder (52), the suture holder distal end (56) is positioned adjacent and inside the tube distal end (34). In the second position of the suture holder (52), the suture holder distal end (56) is extended a distance outwardly from the tube distal end (34).

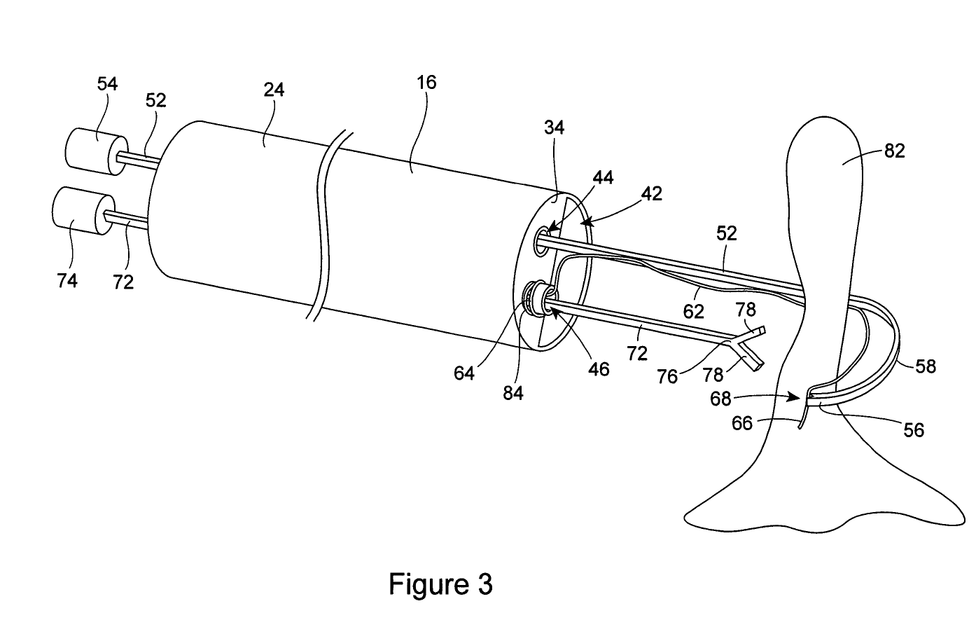

A portion of the suture holder length (58) adjacent the suture holder distal end (56) is bent in a predetermined curve. This portion of the suture holder length (58) is shown in FIGS. 2 and 3. The flexibility of this portion of the suture holder (52) will allow the portion to straighten when the suture holder is moved to its first position relative to the tube (16) and the suture holder distal end (56) is positioned adjacent and inside the tube distal end (34). When the suture holder (52) is moved to its second position relative to the tube (16), the bent portion (58) of the suture holder is extended from the tube distal end (34) and moves to its curved configuration shown. The transverse positioning of the second bore (44) and third bore (46) and the curved configuration of the bent portion (58) are specifically dimensioned to position the suture holder distal end (56) in alignment with and spaced outwardly from the third interior bore (46) of the tube (16) at the tube distal end (34).

The suture holder distal end (56) is provided with a mechanism for removably holding a length of suture (62). The length of suture (62) has opposite proximal (64) and distal (66) ends. As shown in the drawing figures, the suture distal end (66) is removably held by the suture holder distal end (56). The mechanism for holding the suture on the suture holder distal end (56) can be a simple mechanism such as a slot (68) formed into the suture holder distal end (56) with the suture distal end (66) being removably inserted into the slot. Alternatively, the suture holder distal end (56) could be formed with a small hook that hooks around and removably holds the suture holder distal end (66). Other equivalent mechanisms that removably hold the suture distal end (66) to the suture holder distal end (56) could be employed.

A narrow elongate grasper (72) extends through the third interior bore (46) of the tube (16). The grasper (72) has a flexible length that extends between a proximal end (74) and a distal end (76) of the grasper. The grasper length is slightly larger than the length of the endoscope (12) so that the grasper proximal end (74) projects from a port (24) of the control housing (22) with the grasper distal end (76) positioned adjacent and inside the tube distal end (34). The grasper (72) is mounted in the third interior bore (46) of the tube (16) for flexing movement of the grasper length with flexing movement of the tube, and for reciprocating movement of the grasper (72) between first and second positions relative to the tube (16). The grasper (72) can be moved between its first and second positions by a surgeon manipulating the grasper proximal end (74) relative to the control housing (14). Alternatively, a mechanism could be provided on the control housing (14) for reciprocating the length of the grasper (72) through the tube third interior bore (46). When the grasper (72) is in its first position relative to the tube (16), the grasper distal end (76) is positioned adjacent and inside the tube distal end (34). When the grasper (72) is moved to its second position relative to the tube (16), the grasper distal end (76) is extended a distance outwardly from the tube distal end (34).

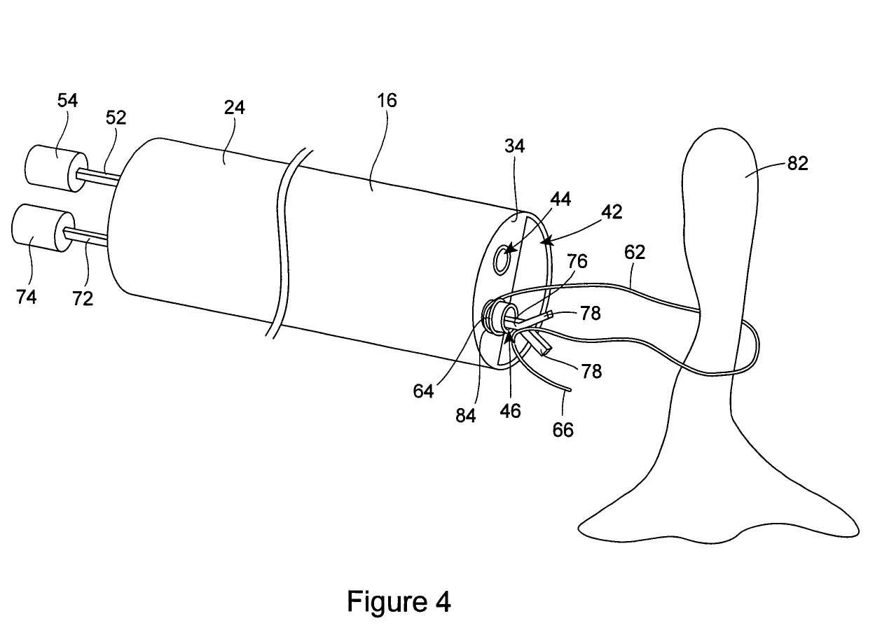

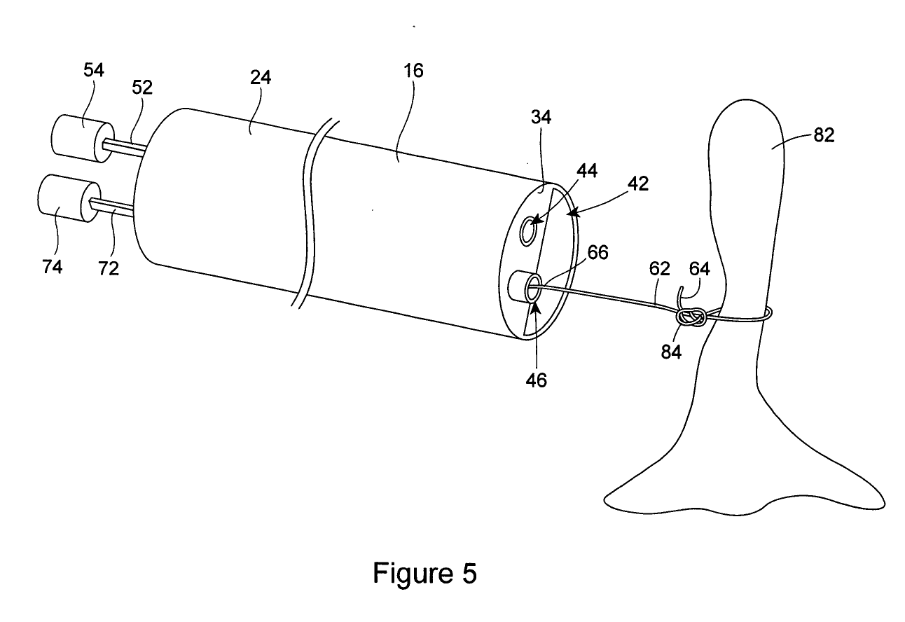

The grasper (72) is provided with a grasping mechanism at the grasper distal end (76). In the embodiment of the invention shown in FIGS. 3 and 4, the grasping mechanism is provided by a pair of jaws (78) at the grasper distal end (76). The grasper jaws (78) can be of any known construction and are operable at the grasper proximal end (74) to selectively open and close the jaws (78). The jaws (78) are dimensioned to close on the suture distal end (66) and remove the suture from the suture holder (68). Other equivalent mechanisms capable of performing these functions could be used in lieu of the grasper jaws (78). For example, a simple hook at the grasper distal end could be used to remove the suture from the suture holder.

In use of the endoscope (12) in performing an endoscopic knot-tying procedure, for example a ligation procedure, the endoscope flexible tube (16) is first inserted through an incision into a body cavity where the procedure is to be performed. In insertion of the endoscope (12) into the body cavity, the tube distal end (34) is positioned proximate the surgical site as represented in FIG. 2. In FIG. 2, the tube distal end (34) is positioned adjacent body tissue (82) that is to be ligated. Although a ligation procedure is described, the endoscope (12) could be employed in practicing other endoscopic suture knot tying procedures.

With the tube distal end (34) positioned adjacent the body tissue (82) to be ligated, the method of using the endoscope (12) in the ligating procedure next involves manipulation of the suture holder (52) to cause the suture holder to be moved from its first position relative to the tube (16), to its second position relative the tube. This is shown in FIG. 2 where the bent portion (58) of the suture holder (52) is extended outwardly from the tube distal end (34). This causes the bent portion (58) to extend around the body tissue (82) to be ligated. At the same time, the length of suture (62) is passed around the body tissue (82) by the suture distal end (66) being removably held by the suture holder distal end (68).

The surgical grasper (72) is then moved from its first position to its second position relative to the endoscope tube (16). This causes the grasper distal end (76) to move to its extended position outwardly from the tube distal end (34). The alignment of the grasper (72) with the suture holder distal end (56) with the suture holder in its second position results in the grasper distal end (76) intersecting the suture holder distal end (56). The jaws (78) on the grasper distal end (76) are positioned to grasp the suture distal end (76). This movement of the grasper is illustrated in FIG. 3.

With the grasper jaws (78) holding the suture distal end (76), the grasper (72) is then moved from its second position back to its first position relative to the tube (16). This causes the grasper distal end (76) to remove the suture distal end (66) from the suture holder distal end (56). The length of suture (62) is pulled around the body tissue (82) as the suture distal end (66) is pulled by the grasper distal end (76) toward the tube distal end (34).

In the preferred embodiment of the apparatus, a portion of the suture adjacent the suture proximal end (64) is tied in a knot (84) around the grasper (72). FIGS. 2-4 show the knot (84) positioned on an extension of the third bore (46) that projects from the tube distal end (34). As the suture distal end (66) is pulled by the grasper distal end (76) toward the tube distal end (34), the suture distal end (66) is pulled through the pre-tied knot (84). This completes a knot tied around the body tissue (82). By continuing to pull the suture distal end (66) through the pre-tied knot (84), the knot in the length of suture (62) is tightened around the body tissue (82), as illustrated in FIG. 5. This results in the ligation of the body tissue (82) using the endoscope (12) of the invention.

Although the endoscope of the invention has been described above with reference to a specific embodiment of the invention, it should be understood that variations and modifications could be made to the endoscope without departing from the intended scope of the following claims.

1. An endoscopic apparatus comprising:
a flexible tube having a narrow elongate length with opposite proximal and distal ends;
a flexible suture holder having a narrow elongate length with opposite proximal and distal ends, the suture holder being mounted to the flexible tube for movement of the suture holder along the tube length between first and second positions of the suture holder relative to the tube where in the first position the suture holder distal end is adjacent the tube distal end and in the second position the suture holder distal end is spaced a distance from the tube distal end; and,
a flexible grasper having a narrow elongate length with opposite proximal and distal ends, the grasper being mounted to the flexible tube for movement of the grasper along the tube length between first and second positions of the grasper relative to the tube where in the first position the grasper distal end is adjacent the tube distal end and in the second position the grasper distal end is spaced a distance from the tube distal end.

2. The apparatus of claim 1, further comprising:
the suture holder and the grasper being mounted to the tube whereby the length of the suture holder and the length of the grasper both flex with flexing of the tube.

3. The apparatus of claim 1, further comprising:
the tube having an exterior surface that extends the length of the tube; and,
the suture holder and the grasper being mounted to the tube inside the tube exterior surface.

4. The apparatus of claim 3, further comprising:
the tube having an interior bore that extends through the tube length along side the suture holder and the grasper with the suture holder and the grasper being outside the tube interior bore.

5. The apparatus of claim 3, further comprising:
the suture holder and the grasper being movable along the tube length and being held by the tube against movement transverse to the tube length.

6. The apparatus of claim 1, further comprising:
the suture holder having a distal end portion that projects in a curved configuration from the tube distal end when the suture holder is in the second position of the suture holder relative to the tube; and,
the grasper having a distal end portion that projects in a straight configuration from the tube distal end when the grasper is in the second position of the grasper relative to the tube.

7. The apparatus of claim 6, further comprising:
the curved configuration of the suture holder distal end portion positioning the suture holder distal end in alignment with the grasper distal end when the suture holder and the grasper are in their second positions relative to the tube.

8. The apparatus of claim 7, further comprising:
a length of suture having opposite proximal and distal ends; and,
the suture holder distal end removably holding the length of suture distal end.

9. The apparatus of claim 8, further comprising:
the length of suture proximal end having a knot.

10. An endoscope apparatus comprising:
a flexible tube having a narrow elongate length with opposite proximal and distal ends;
a flexible suture holder having a narrow elongate length with opposite proximal and distal ends, the suture holder being mounted inside the tube for flexing movement of the suture holder with flexing movement of the tube, and for movement of the suture holder along the tube length between first and second positions of the suture holder relative to the tube where in the first position of the suture holder the suture holder distal end is adjacent the tube distal end and in the second position of the suture holder the suture holder distal end is extended a distance out from the tube distal end; and,
a flexible grasper having a narrow elongate length with opposite proximal and distal ends, the grasper being mounted inside the tube for flexing movement of the grasper with flexing movement of the tube, and for movement of the grasper along the tube length between first and second positions of the grasper relative to the tube where in the first position of the grasper the grasper distal end is positioned adjacent the tube distal end and in the second position of the grasper the grasper distal end is extended a distance out from the tube distal end.

11. The apparatus of claim 10, further comprising:
the tube having an interior bore extending through the tube length for containing endoscopic devices in the interior bore; and,
the suture holder and the grasper being separate from the interior bore and extending along side the interior bore.

12. The apparatus of claim 11, further comprising:
the tube having a suture holder bore extending through the tube length along side the tube interior bore;
the suture holder being mounted in the suture holder bore for reciprocating movement of the suture holder in the suture holder bore;
the tube having a grasper bore extending through the tube length along side the tube interior bore; and,
the grasper being mounted in the grasper bore for reciprocating movement of the grasper in the grasper bore.

13. The apparatus of claim 12, further comprising:
the suture holder having a distal end portion that projects in a curved configuration from the tube distal end when the suture holder is in the second position of the suture holder relative to the tube; and,
the grasper having a distal end portion that projects in a straight configuration from the tube distal end when the grasper is in the second position of the grasper relative to the tube.

14. The apparatus of claim 13, further comprising:
the curved configuration of the suture holder distal end portion positioning the suture holder distal end in alignment with the grasper distal end when the suture holder and the grasper are in their second positions relative to the tube.

15. The apparatus of claim 14, further comprising:
a length of suture having opposite proximal and distal ends; and,
the suture holder distal end removably holding the length of suture distal end.

16. The apparatus of claim 15, further comprising:
the length of suture proximal end having a knot.

17. The apparatus of claim 16, further comprising:
the knot being tied around the grasper.

18. An endoscope apparatus comprising:
a flexible tube having a length with opposite proximal and distal ends;
a first interior bore extending through the length of the tube;
a second interior bore extending through the length of the tube, the second interior bore being separated from the first interior bore;
a third interior bore extending through the length of the tube, the third interior bore being separated from the first and second interior bores;
a flexible suture holder having a length with opposite proximal and distal ends, the length of the suture holder being mounted in the tube second interior bore for flexing movement of the suture holder with flexing movement of the tube and for reciprocating movement of the suture holder through the tube second interior bore between first and second positions of the suture holder relative to the tube where in the first position the suture holder distal end is positioned adjacent the tube distal end and in the second position the suture holder distal end is positioned at a spaced distance from the tube distal end;
a length of suture having opposite proximal and distal ends, the suture distal end being removably held by the suture holder distal end; and,
a flexible grasper having a length with opposite proximal and distal ends, the length of the grasper being mounted in the tube third interior bore for flexing movement of the grasper with flexing movement of the tube and for reciprocating movement of the grasper through the tube third interior bore between first and second positions of the grasper relative to the tube where in the first position the grasper distal end is positioned adjacent the tube distal end and in the second position the grasper distal end is positioned at a spaced distance from the tube distal end;
a distal end portion of the suture holder having a bent configuration that bends into a curve as the suture holder is moved from the first position to the second position of the suture holder relative to the tube, the bent configuration of the suture holder distal end portion positioning the suture holder distal end in alignment with the grasper distal end with the grasper in the second position, whereby the grasper distal end in the second position of the grasper is operative to grasp the suture held by the suture holder distal end in the second position of the suture holder and remove the suture from the suture holder.

19. The apparatus of claim 18, further comprising:
the first bore being dimensioned for containing at least one endoscopic device in the first bore separated from the suture holder and the grasper.

20. The apparatus of claim 18, further comprising:
the length of suture having a knot adjacent the suture proximal end.
